(19) United States
(12) Patent Application Publication
(10) Pub. No.: US 2024/0099873 A1
(43) Pub. Date: Mar. 28, 2024

(54) PELVIC ORTHODONTIC DEVICE

(71) Applicant: ZAN-YU ZENG, New Taipei (TW)

(72) Inventor: ZAN-YU ZENG, New Taipei (TW)

(21) Appl. No.: 18/372,142

(22) Filed: Sep. 25, 2023

(30) Foreign Application Priority Data
Sep. 27, 2022 (TW) 111136787

(51) Int. Cl. A61F 5/02

(52) U.S. Cl. CPC A61F 5/028

(57) ABSTRACT

The present invention relates to a pelvic orthodontic device, comprises a device body, on which there are three sacral vertebra support portions, a lever fulcrum, and three direction guiding fulcrums. The sacral vertebra support portions and the lever fulcrum are formed at the position relative to a sacral vertebra of the human body. Three sacral vertebra support portions, the lever fulcrum and the device body which have a height difference shape. When the sacral vertebra of the human body is in contact with the orthodontic device, the three sacral vertebra support portions form a three-point tripod and interact with the lever fulcrum to adjust the displacement of the sacral vertebra caused by injury and correct the problem of pelvic rotation. Avoiding the compensation of bad movement or posture, so as to avoid suffering from chronic pain.

### BACKGROUND OF THE INVENTION

### Field of the Invention

The present invention discloses a device for correcting human pelvic displacement, especially a device designed under the sacral vertebra and capable of correcting the pelvis.

### The Prior Arts

Along with the development and progress of the economy, the chances of performing physical labor in modern life are greatly reduced than in the past, which leads to the lack of exercise of modern people and makes such as dysmetabolic syndrome, obesity, hypertension, hyperlipidemia, and low cardiopulmonary function. Such that the disease of civilization to continue unabated. Adding to these diseases of civilization is the common and pervasive form of chronic pain. The main cause of this chronic pain is that poor movement and posture make the musculoskeletal balance unbalanced. Displacement of the sacral vertebra of the pelvis due to a fall that is the biggest reason, because the displacement of the pelvis will have the effect of replacing and compensating, allowing the human body to continue to operate in a bad state.

However, the above-mentioned action of replacing compensation is an interaction between the skeletal muscles in different positions. This interaction between the skeletal muscles may cause various undesirable actions and movements when people are sitting or standing. The bad postures and bad movements cause excessive use of some skeletal muscles, which in turn leads to substitution and compensation under bad movements and postures, causing the aforementioned chronic pain and various aches and pains.

Since the above-mentioned chronic pain partly comes from the overall replacement and compensation of the human body, it is difficult for the medical technology or scientific technology in the related field to easily prescribe the right medicine or solve the problem in a targeted manner, that is, the existing medical technology or scientific technology, however, there is still no specific and simple and feasible means to maintain the bones, muscles and joints of the body in a balanced state at any time in daily life, and there is no way to effectively prevent bad movements and postures from causing soreness and the aforementioned chronic pain.

When human beings stand or walk, the most ideal state is that the pelvis can maintain a balanced position, and the injury and pelvic displacement caused by falling will cause other parts of the body to lose balance.

In view of the above problems, in the past, medical personnel used manual treatment to improve the treatment method, and there is no effective device to assist medical personnel, and it is impossible to effectively deal with the problem of sacral vertebral displacement in large quantities.

The displacement of the sacral vertebra needs to be solved effectively with the help of appropriate methods. The market has its demand for devices for pelvic correction, but currently there is no product that can meet this demand on the market. Therefore, how to provide the product that can satisfy this demand is a problem to be solved extremely.

In view of the fact that the aforementioned prior art cannot effectively solve the problem of displacement of the sacral vertebra, and the market demand for related products, the main purpose of the present invention is to provide a device for correcting the pelvis.

How to improve the above-mentioned unbalanced state is that when the human being lies down, the sacral vertebra is properly changed when it contacts the orthotic device. Under natural conditions, the sacral vertebrae are supported by three support positions when lying down, which can change the displacement of the sacral vertebrae due to falls and balance the pelvic position. In this way, the body is naturally balanced, and bad movements and postures of various parts of the body are also changed.

### SUMMARY OF THE INVENTION

In order to achieve the above-mentioned purpose of the invention, the main technical means used in the present invention is to make the design of the aforementioned pelvic orthodontic device, comprising a device body, three sacral vertebra support portions protruding from the aforementioned device body, a lever fulcrum, direction guiding fulcrums, and there is a height difference between the device body and the sacral vertebra support portion and the lever fulcrum. The pelvic orthodontic device can also be reversed, and has the same effect.

Because the present invention has the above-mentioned structure, the sacral vertebra support portion can be formed into shapes such as arc surface or plane. Generally speaking, the aforementioned height difference causes the contact force between the sacral vertebra of the human body and the device body to change. The function of three position stand and lever fulcrum can change the problem of displacement of sacral vertebra, avoid problems caused by imbalance of joints in the whole body, and thus avoid suffering from chronic pain.

### BRIEF DESCRIPTION OF THE DRAWINGS

FIG. 1 is a top view of the first embodiment of the present invention.

FIG. 2 is a sectional view of the second embodiment of the present invention.

FIG. 3 is a sectional view of the third embodiment of the present invention.

FIG. 4 is a schematic side view of the present invention implemented in a lying position.

### DETAILED DESCRIPTION OF THE PREFERRED EMBODIMENT

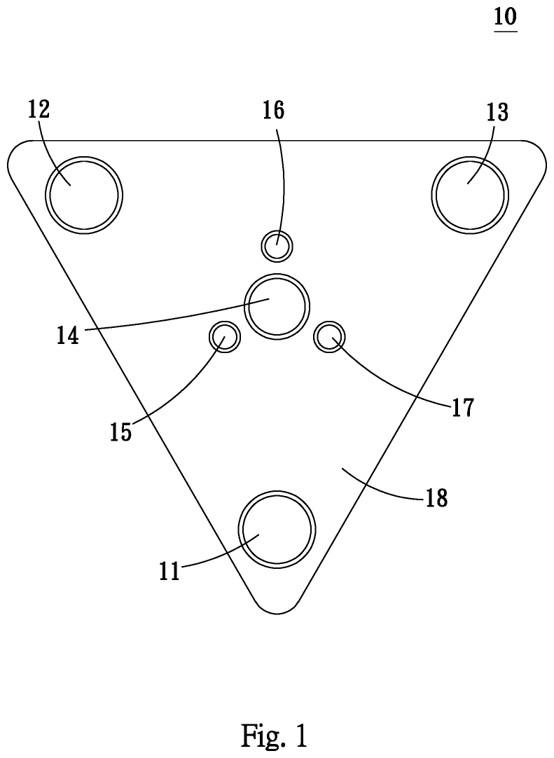

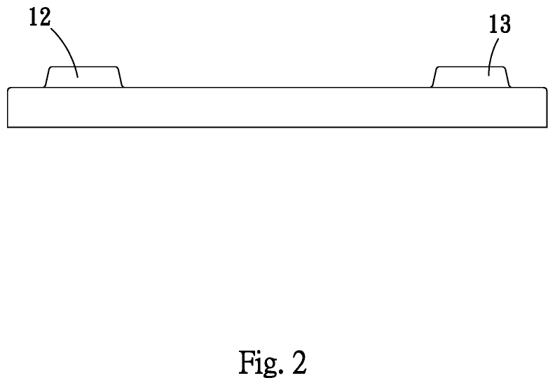

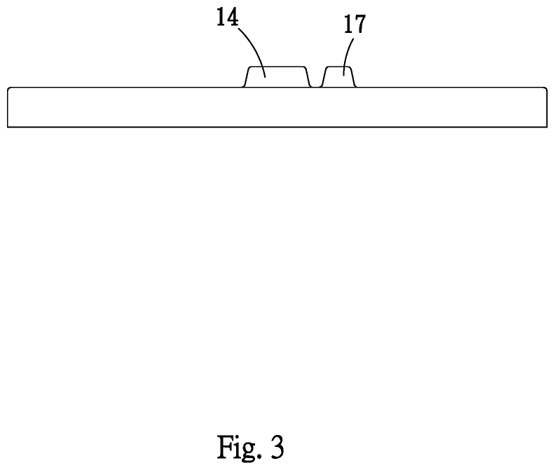

Please refer to FIG. 1 and FIG. 2, FIG. 3, the present invention is a kind of pelvic orthodontic device, its first embodiment comprises a device body (10), a sacral vertebra support portion (11), (12), (13) on the device body (10), a lever fulcrum (14), three directions guide the fulcrum (15) (16) (17), a height difference portion (18).

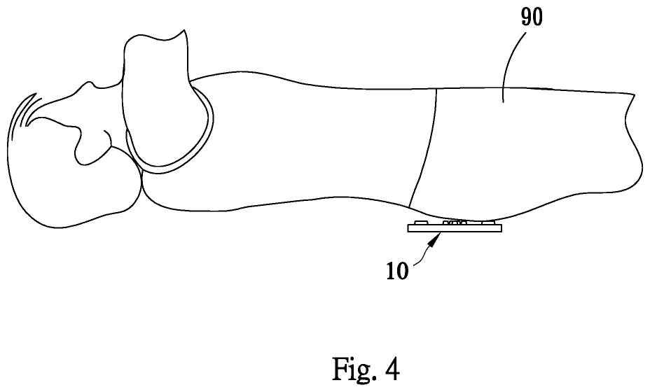

Please refer to FIG. 4. When the aforementioned device body (10) is used for lying down, forming a cushion placed under the sacral vertebra of the pelvis. The sacral vertebra of the displacement is automatically adjusted by three portion stand principle and the fulcrum of the lever. The device body (10) contains three sacral vertebra support portions (11), (12), (13), a lever fulcrum (14), three direction guiding fulcrums (15) (16) (17), a height difference portion (18), when the human body (90) lies on the device body (10), the acral vertebra support portions (11), (12), (13) produce three portion stand contact force, and cooperate with the lever fulcrum (14), gradually improve the displacement of the sacral vertebrae due to falls and injuries, correct the problem of pelvic rotation, avoid compensation due to bad movements or postures, and avoid suffering from chronic pain, that is, good results will be obtained.

The device body (10) can be made on the uppermost layer of any shape lying movable pad for use when lying down.

The device of the present invention supports the sacral vertebra support portion (11) (12) (13), the lever fulcrum (14) and the three direction guiding fulcrums (15) (16) (17), and a height difference portion (18). It can automatically adjust the pelvis, allowing the body to naturally return to a state of balance.

With the pelvic orthodontic device provided by the present invention, the rotation of the pelvis can be adjusted so that the musculoskeletal system reaches a balanced position and reaches the most ideal state. The musculoskeletal system of the human body is continuously maintained in an ideal state of balance by using the present invention. It can reduce possible injuries caused by skeletal and muscular imbalances, improve people's soreness and chronic pain, make people feel comfortable all over the body and avoid injuries caused by bad movements or postures.

What is claimed is:

1. A pelvic orthodontic device, comprising:
a device body having a thickness;
three sacral support portions being formed on the device body for correction.

2. The pelvic orthodontic device as claimed in claim 1, wherein the pelvic orthodontic device comprises a lever fulcrum which is located among a middle of the said three sacral vertebra support portions.

3. The pelvic orthodontic device as claimed in claim 1, wherein the pelvic orthodontic device has three direction guiding fulcrum therein.

4. The pelvic orthodontic device as claimed in claim 1, wherein the pelvic orthodontic device can be reversed downward.
